(19) United States
(12) Patent Application Publication
(10) Pub. No.: US 2021/0382250 A1
(43) Pub. Date: Dec. 9, 2021

(54) MANUFACTURING METHOD FOR IMAGE PICKUP APPARATUS FOR ENDOSCOPE, IMAGE PICKUP APPARATUS FOR ENDOSCOPE, AND ENDOSCOPE

(71) Applicant: OLYMPUS CORPORATION, Tokyo (JP)

(72) Inventor: Takashi NAKAYAMA, Ina-shi (JP)

(73) Assignee: OLYMPUS CORPORATION, Tokyo (JP)

(21) Appl. No.: 17/411,710

(22) Filed: Aug. 25, 2021

Related U.S. Application Data
(63) Continuation of application No. PCT/JP2019/017048, filed on Apr. 22, 2019.

(51) Int. Cl. G02B 6/42; G02B 23/26; H04N 5/225

(52) U.S. Cl. CPC G02B 6/4239; G02B 23/26; A61B 1/00165; H04N 5/2254; H04N 2005/2255; G02B 6/4243

(57) ABSTRACT

A manufacturing method for an image pickup apparatus for endoscope includes forming a plurality of insertion holes in a first wafer and forming, in a second wafer, a plurality of through-holes including guide surfaces, bonding the first wafer and the second wafer to produce a bonded wafer, cutting the bonded wafer to thereby produce ferrules to which guide members are bonded, and mounting an optical element on each of the ferrules and fixing optical fibers with resin in a state in which the optical fibers are inserted into the insertion holes by passing through the guide members.

### CROSS REFERENCE TO RELATED APPLICATION

This application is a continuation application of PCT/JP2019/017048 filed on Apr. 22, 2019, the entire contents of which are incorporated herein by this reference.

### BACKGROUND OF THE INVENTION

### 1. Field of the Invention

The present invention relates to a manufacturing method for an image pickup apparatus for endoscope including an image pickup device, an optical element, and an optical fiber, an image pickup apparatus for endoscope including an image pickup device, an optical element, and an optical fiber, and an endoscope including an image pickup apparatus for endoscope including an image pickup device, an optical element, and an optical fiber.

### 2. Description of the Related Art

An endoscope includes an image pickup apparatus at a distal end portion of an elongated insertion section. In recent years, an image pickup device having a large number of pixels has been studied in order to display a high-quality image. In an image pickup apparatus in which the image pickup device having a large number of pixels is used, the amount of image signals transmitted to a signal processing apparatus (a processor) increases.

In order to reduce a diameter of the insertion section to make the insertion section less invasive, optical signal transmission through a thin optical fiber by an optical signal instead of an electric signal is preferable. For the optical signal transmission, an E/O type optical module (an electrooptical converter) that converts an electric signal into an optical signal and an O/E type optical module (a photoelectric converter) that converts an optical signal into an electric signal are used.

Japanese Patent Application Laid-Open Publication No. 20134025092 discloses an optical module including an optical element, a substrate on which the optical element is mounted, and a holding unit (a ferrule) including a through-hole into which an optical fiber for transmitting an optical signal inputted to or outputted from the optical element is inserted.

Japanese Utility Model Application Laid-Open Publication No. S60-41915 discloses an optical terminal block in which a semicircular groove extends from a circular insertion hole into which an optical fiber is inserted. The optical fiber disposed in the groove is fixed by a pressing member in which a semicircular groove is formed.

### SUMMARY OF THE INVENTION

A manufacturing method for an image pickup apparatus for endoscope in an embodiment includes: forming, in a first wafer, a plurality of insertion holes into which a plurality of optical fibers are individually inserted and forming, in a second wafer, a plurality of through-holes each including, on a wall surface, a guide surface functioning as a guide in inserting the plurality of optical fibers individually into respective holes of the plurality of insertion holes; bonding the first wafer and the second wafer to produce a bonded wafer; cutting the bonded wafer to thereby produce ferrules to which guide members each including the guide surface are bonded; and mounting an optical element on each of the ferrules and fixing the optical fibers with resin in a state in which the optical fibers are inserted into the insertion holes by passing through the respective guide members.

An image pickup apparatus for endoscope in an embodiment includes: an image pickup device configured to output an image pickup signal; an optical element configured to convert the image pickup signal into an optical signal; an optical fiber configured to transmit the optical signal; a ferrule including a first principal surface and a second principal surface on an opposite side of the first principal surface, the optical element being mounted on the first principal surface, the optical fiber being inserted into an insertion hole with an opening on the second principal surface; and a guide member including a third principal surface and a fourth principal surface on an opposite side of the third principal surface, the third principal surface being bonded to the second principal surface, the guide member including a guide surface extending from an inner surface of the insertion hole.

An endoscope in an embodiment includes an image pickup apparatus for endoscope including: an image pickup device configured to output an image pickup signal; an optical element configured to convert the image pickup signal into an optical signal; an optical fiber configured to transmit the optical signal; a ferrule including a first principal surface and a second principal surface on an opposite side of the first principal surface, the optical element being mounted on the first principal surface, the optical fiber being inserted into an insertion hole with an opening on the second principal surface; and a guide member including a third principal surface and a fourth principal surface on an opposite side of the third principal surface, the third principal surface being bonded to the second principal surface, the guide member including a guide surface extending from an inner surface of the insertion hole.

### BRIEF DESCRIPTION OF THE DRAWINGS

FIG. 1 is a perspective view of an endoscope system including an endoscope including an image pickup apparatus for endoscope in an embodiment:

FIG. 2 is a perspective view of the image pickup apparatus for endoscope in the embodiment;

FIG. 3 is a sectional view taken along a III-III line in FIG. 2;

FIG. 4 is an exploded view of the image pickup apparatus for endoscope in the embodiment;

FIG. 5 is a flowchart of a manufacturing method for the image pickup apparatus for endoscope in the embodiment;

FIG. 6 is a sectional view for explaining the manufacturing method for the image pickup apparatus for endoscope in the embodiment;

FIG. 7 is a sectional view for explaining the manufacturing method for the image pickup apparatus for endoscope in the embodiment;

FIG. 8 is a perspective view for explaining the manufacturing method for the image pickup apparatus for endoscope in the embodiment;

FIG. 9 is a perspective view for explaining the manufacturing method for the image pickup apparatus for endoscope in the embodiment;

FIG. 10 is a perspective view for explaining the manufacturing method for the image pickup apparatus for endoscope in the embodiment;

FIG. 11 is an exploded view of an image pickup apparatus for endoscope in a modification 1;

FIG. 12A is a plan view of a guide member of an image pickup apparatus for endoscope in a modification 2;

FIG. 12B is a plan view of a guide member of an image pickup apparatus for endoscope in a modification 3;

FIG. 13 is a sectional view of an image pickup apparatus for endoscope in a modification 4; and

FIG. 14 is a sectional view of an image pickup apparatus for endoscope in a modification 5.

### DETAILED DESCRIPTION OF THE PREFERRED EMBODIMENTS

### <Endoscope>

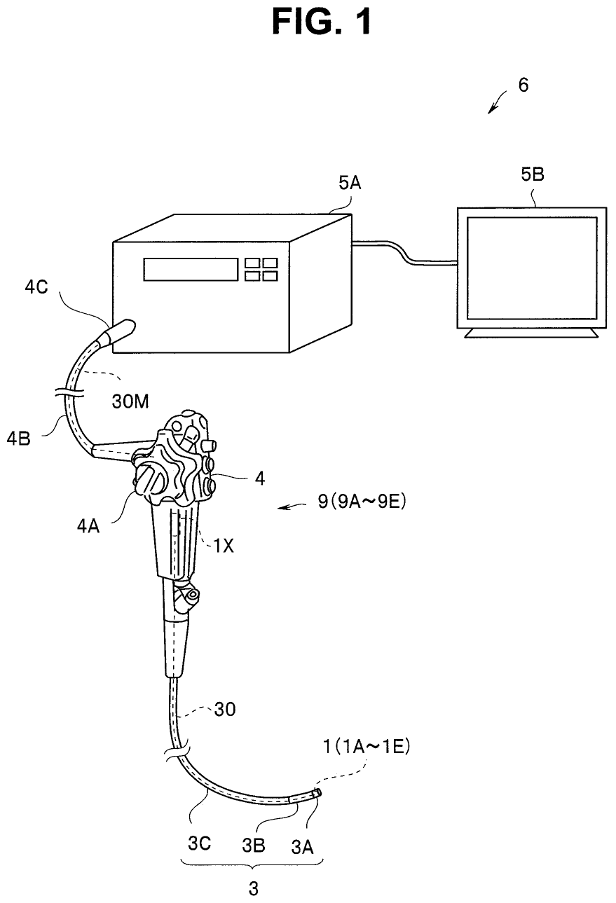

An endoscope 9 in an embodiment shown in FIG. 1 configures an endoscope system 6 in conjunction with a processor 5A and a monitor 5B.

The endoscope 9 includes an insertion section 3, a grasping section 4 disposed at a proximal end portion of the insertion section 3, a universal cord 4B extending from the grasping section 4, and a connector 4C disposed at a proximal end portion of the universal cord 4B. The insertion section 3 includes a distal end portion 3A, a bending portion 3B for changing a direction of the distal end portion 3A, the bending portion 3B extending from the distal end portion 3A and being bendable, and a flexible portion 3C extending from the bending portion 3B. A turning angle knob 4A, which is an operation section for a surgeon to operate the bending portion 3B, is disposed in the grasping section 4.

The universal cord 4B is connected to the processor 5A by the connector 4C. The processor 5A controls the entire endoscope system 6, performs signal processing on an image pickup signal, and outputs an image signal. The monitor 5B displays, as an endoscopic image, the image signal outputted by the processor 5A. Note that the endoscope 9 is a flexible endoscope but may be a rigid endoscope. The endoscope 9 may be either for medical use or for industrial use.

An image pickup apparatus 1 is disposed at the distal end portion 3A of the endoscope 9. The image pickup apparatus 1 outputs an image pickup signal as an optical signal. The optical signal is converted into an electric signal again by an O/E type optical module 1X disposed in the grasping section 4 by passing through an optical fiber 30 inserted through the insertion section 3 and is transmitted by passing through a metal wire 30M. In other words, the image pickup signal is transmitted by passing through the optical fiber 30 in the small-diameter insertion section 3 and is transmitted by passing through a signal cable 30M, which is a metal wire thicker than the optical fiber 30, in the universal cord 4B not inserted into a body and having small limitation of an outer diameter.

Note that when an O/E type optical module is disposed in the connector 4C or the processor 5A, the optical fiber 30 is inserted through the universal cord 4B.

As explained below, the image pickup apparatus 1 is small and has high reliability. Accordingly, the endoscope 9 is less invasive and has high reliability.

### <Image Pickup Apparatus>

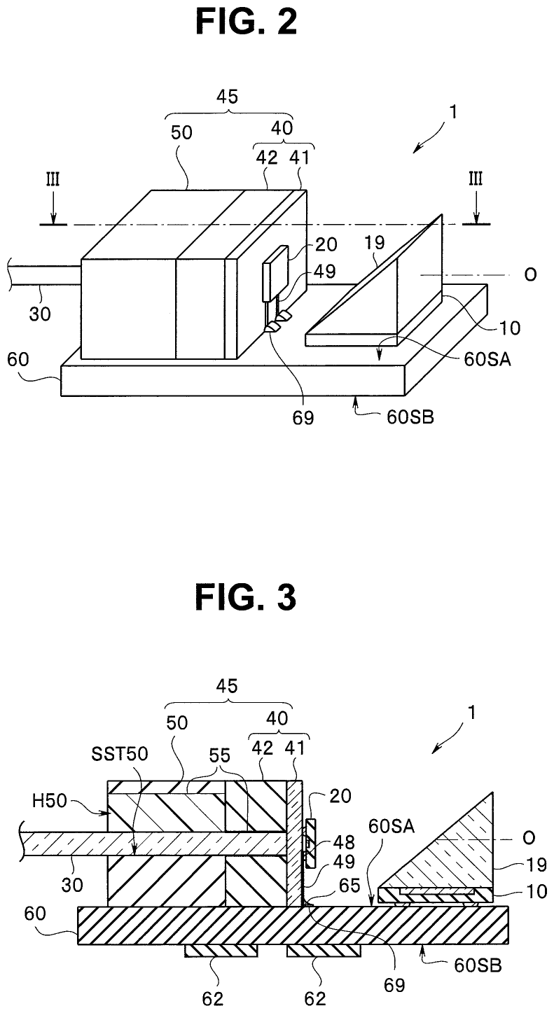

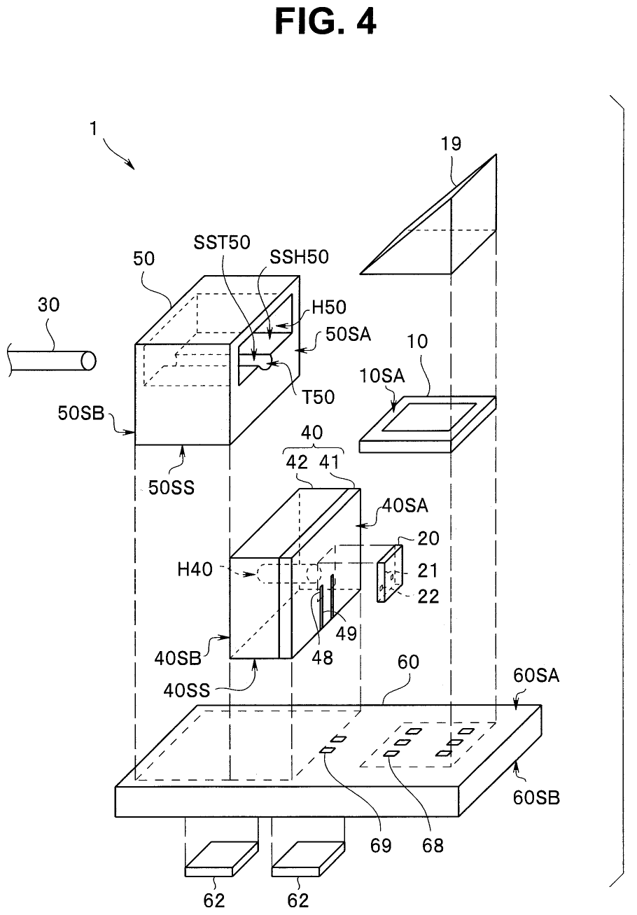

The image pickup apparatus 1 in the embodiment is shown in FIG. 2, FIG. 3, and FIG. 4. The image pickup apparatus 1 in the embodiment includes an image pickup device 10, an optical element 20, an optical fiber 30, a ferrule 40, a guide member 50, and a wiring board 60.

In the following explanation, drawings based on respective embodiments are schematic. Relations between thicknesses and widths of respective portions, ratios of the thicknesses and relative angles of the respective portions, and the like are different from real ones. Portions, relations and ratios of dimensions of which are different, are included among the drawings. Illustration of a part of constituent elements is omitted. A direction in which the image pickup device 10 is disposed in an optical axis direction of the image pickup apparatus 1 is referred to as “front” and a direction in which the optical fiber 30 extends is referred to as “rear”.

The image pickup device 10 includes a light receiving unit including a CCD or CMOS image pickup unit, receives an object image, and outputs an image pickup signal. The image pickup device 10 may be either a surface irradiation type image sensor or a rear surface irradiation type image sensor. The image pickup apparatus 1 is a so-called horizontal type in which a light receiving surface of the image pickup device 10 is parallel to an optical axis of an optical system (not shown). In the image pickup apparatus 1, an object image condensed by the optical system is made incident on the image pickup device 10 by passing through a prism 19.

The optical element 20 is a light-emitting element that converts an image pickup signal into an optical signal. For example, the ultrasmall optical element 20, a plan view dimension (an external dimension of a cross section orthogonal to an optical axis O) of which is 235 μm×235 μm, includes a light emitting unit 21, a diameter of which is 10 μm, and an external electrode 22, a diameter of which is 70 μm, that supplies a driving signal to the light emitting unit 21. Note that the image pickup signal outputted by the image pickup device 10 is processed by electronic components 62 such as a drive 1C and then inputted to the optical element 20.

The optical fiber 30 transmits an optical signal emitted by the optical element 20. The optical fiber 30 includes, for example, a 62.5 μm-diameter core that transmits light and an 80 μm-diameter clad that covers an outer circumferential surface of the core.

The ferrule 40 includes a first principal surface 40SA and a second principal surface 40SB on an opposite side of the first principal surface 40SA. A glass plate 41 configuring the first principal surface 40SA and a silicon plate 42 configuring the second principal surface 40SB are bonded, whereby the ferrule 40 is produced. The ferrule 40 is a rectangular parallelepiped, an optical axis direction dimension of which is 500 μm.

A first bonding electrode 48 and a wire 49 extending from the first bonding electrode 48 are disposed on the first principal surface 40SA of the ferrule 40. The external electrode 22 of the optical element 20 is bonded to the first bonding electrode 48. In other words, the optical element 20 is mounted on the first principal surface 40SA.

On the other hand, in the ferrule 40, an opening is present in the second principal surface 40SB and an insertion hole H40 including the glass plate 41 as a bottom surface is formed. The optical fiber 30 is inserted into the insertion hole H40.

The guide member 50 includes a third principal surface 50SA and a fourth principal surface 50SB on an opposite side of the third principal surface 50SA. The third principal surface 50SA is bonded to the second principal surface 40SB of the ferrule 40. The guide member 50 includes a guide surface SST50 functioning as a guide when the optical fiber 30 is inserted into the insertion hole H40 of the ferrule 40.

A through-hole H50 piercing from the third principal surface 50SA to the fourth principal surface 50SB is formed in the guide member 50. The guide surface SST50 is an inner surface of a semicircular groove T50 formed on a wall surface SSH50 of the through-hole H50 and is a surface with which the optical fiber 30 is in contact. Note that a cross section of the groove T50 is not limited to the semicircular shape if the groove T50 has a concave shape that can guide the optical fiber 30 and may be a triangular shape or may be a polygonal shape. Width of the groove T50 is the same as an inner diameter of the insertion hole H40. Depth of the groove T50 is a half of the inner diameter of the insertion hole H40. The guide member 50 is disposed in such a position that the guide surface SST50 extends from the inner surface of the insertion hole H40.

Resin 55 that fixes the optical fiber 30 to the insertion hole H40 is injected into the through-hole H50 as well. As the resin 55, various kinds of resin having high light transmittance and a predetermined refractive index, for example, silicone resin, acrylic resin, or epoxy resin is used.

The image pickup device 10, the ferrule 40 (45) to which the guide member 50 is bonded, and the electronic components 62 are disposed on the wiring board 60. An external electrode of the image pickup device 10 is bonded to an electrode 68 on a front surface 60SA of the wiring board 60. An end portion of the wire 49 of the ferrule 40 is bonded to a second bonding electrode 69 on the front surface 60SA by solder 65. The electronic components 62 are mounted on a rear surface 60SB. Note that the end portion of the wire 49 and the second bonding electrode 69 may be electrically connected using a conductive adhesive instead of the solder.

The image pickup apparatus 1 is ultrasmall, for example, 1 mm square in an external dimension in an optical axis orthogonal direction of the ferrule 40. However, since the guide member 50 is bonded to the ferrule 40, it is easy to insert the optical fiber 30. The ferrule 40 to which the guide member 50 is bonded is manufactured by cutting a bonded wafer. Accordingly, the endoscope 9 including the image pickup apparatus 1 is less invasive and is easily manufactured.

### <Manufacturing Method for the Image Pickup Apparatus>

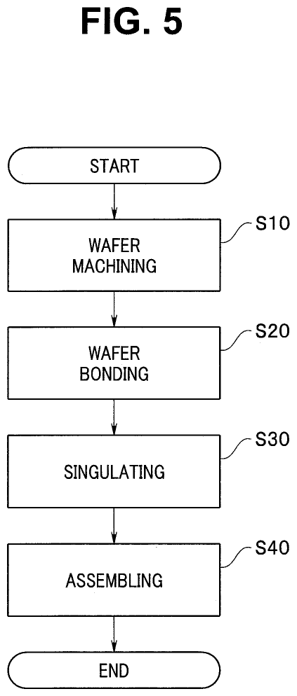

A manufacturing method for the image pickup apparatus 1 is explained with reference to a flowchart of FIG. 5.

### <Step S10>> Wafer Machining Step

In a bonded wafer 40W, a plurality of insertion holes H40 into which a plurality of optical fibers 30 are individually inserted are formed. The wafer machining step includes a glass/silicon bonding step for producing a first wafer (a first bonded wafer).

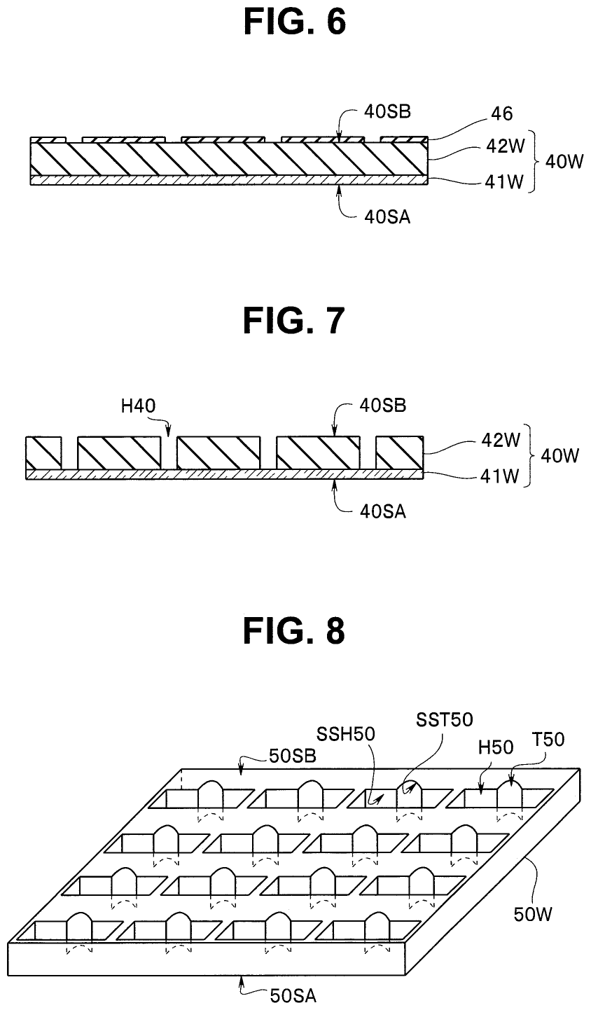

In other words, as shown in FIG. 6, a first silicon wafer 42W and a glass wafer 41W are anodically bonded, whereby a glass/silicon bonded wafer (a first wafer) 40W is produced. An etching mask 46 is disposed on the second principal surface 40SB of the bonded wafer 40W.

As shown in FIG. 7, dry etching treatment such as RIE is performed, whereby a plurality of insertion holes H40 are formed. Since the glass wafer 41W functions as an etching stop layer, depth of the bottomed insertion hole H40 is the same as thickness of the first silicon wafer 42W.

The insertion hole H40 may be formed not by the dry etching but by wet etching. An inner surface shape of the insertion hole H40 may be a prism shape other than a columnar shape if the optical fiber 30 can be held by the inner surface of the insertion hole H40.

Note that when the glass wafer 41W is thick, the insertion hole H40 may be formed after the glass wafer 41W is machined into a thin layer that sufficiently transmits light having a wavelength of an optical signal. Thickness of the glass wafer 41W is preferably 50 μm or less. If the thickness of the glass wafer 41W is 5 μm or more, the glass wafer 41W is less easily broken. The first bonding electrode 48 and the wire 49 made of, for example, gold are disposed on the first principal surface 40SA of the glass wafer 41W using a sputter method.

On the other hand, as shown in FIG. 8, a plurality of through-holes H50 are formed in a second silicon wafer 50W, which is a second wafer. The plurality of through-holes H50 include, on wall surfaces SSH50, guide surfaces SST50 functioning as guides when the plurality of optical fibers 30 are individually inserted into respective holes of the plurality of insertion holes H40.

In other words, an etching mask is disposed on the second silicon wafer 50W including the third principal surface 50SA and the fourth principal surface 50SB on the opposite side of the third principal surface 50SA and etching treatment is performed, whereby the plurality of through-holes H50 are formed. The guide surface SST50 is an inner surface of the groove T50 of the wall surface SSH50. The through-hole H50 is substantially rectangular.

### <Step S20> Wafer Bonding Step

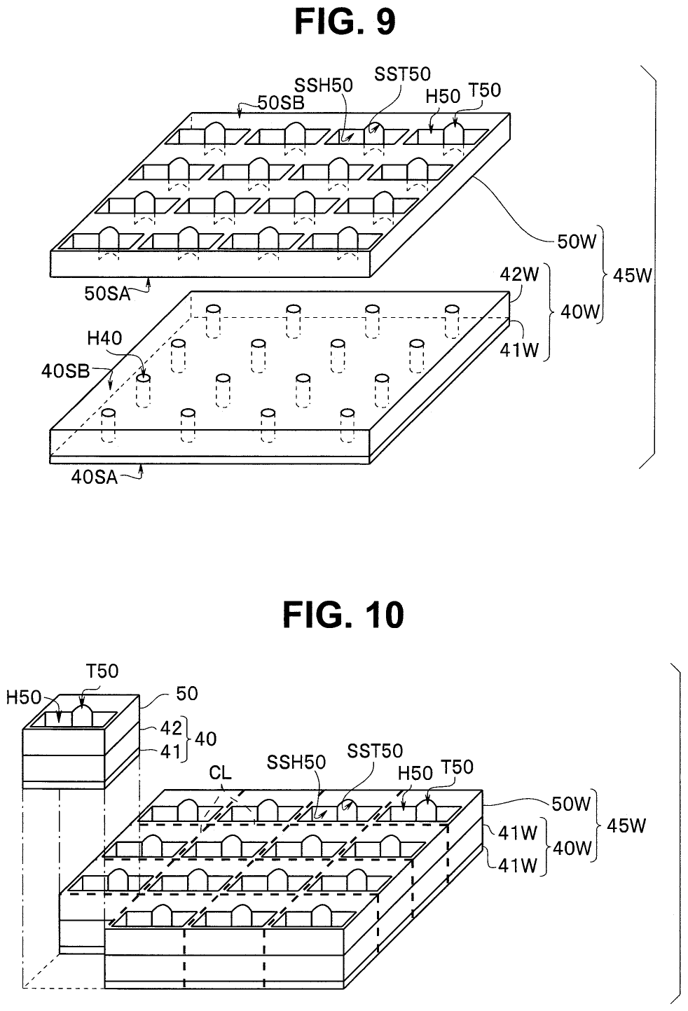

As shown in FIG. 9, a bonded wafer (a second bonded wafer) 45W is produced by bonding the glass/silicon bonded wafer (the first wafer) 40W (41W, 42W) and the second silicon wafer 50W.

The insertion hole H40 of the bonded wafer 40W and the groove T50 of the second silicon wafer 50W are aligned in a state in which the inner surface of the groove T50 extends from the inner surface of the insertion hole H40.

### <Step S30> Singulating Step

As shown in FIG. 10, ferrules 40 (45) to which guide members 50 each including the guide surface SST50 are bonded are produced by cutting the bonded wafer 45W.

In the ultrasmall image pickup apparatus, it is not easy to dispose the guide members 50 in predetermined positions of the ferrules 40. It is difficult to align the singulated ferrules 40 and the guide members 50 to be in a predetermined positional relation because the ferrules 40 are small. It is necessary to further align each set of the ferrule 40 and the guide member 50.

However, in the image pickup apparatus 1, ferrules can be easily produced by cutting the bonded wafer 45W. For example, when the bonded wafer 45W is produced by bonding the wafer 50W including a plurality of ferrules and the wafer 40W including a plurality of guide members, all the ferrules and all the guide members are in a predetermined positional relation if the ferrules and the guide members are aligned to be in the predetermined positional relation in two places. By cutting the bonded wafer 45W, it is possible to easily produce a plurality of ferrules attached with guide members in which the ferrules and the guide members are in the predetermined positional relation.

### <Step S40> Assembling Step

The optical element 20 is mounted on the ferrule 40. The image pickup device 10 and the ferrule 40 to which the guide member 50 is bonded are mounted on the wiring board 60. The external electrode (not shown) of the image pickup device 10 is bonded to the electrode 68 on the front surface 60SA of the wiring board 60. The ferrule 40 to which the guide member 50 is bonded is bonded to the front surface 60SA by an adhesive (not shown). The end portion of the wire 49 of the ferrule 40 is bonded to the second bonding electrode 69 on the front surface 60SA by solder.

The optical fiber 30 is inserted into the insertion hole H40 by passing through the guide member 50 and fixed by the resin 55. For example, the resin 55 is ultraviolet curing resin.

When the optical fiber 30 is inserted into the insertion hole H40 into which the resin 55 is injected, a part of the resin 55 overflows to the through-hole H50 of the guide member 50. In the image pickup apparatus 1, the resin 55 overflowing the insertion hole H40 is stored in the through-hole H50. Therefore, the resin 55 does not spread to a periphery.

The optical fiber 30 is stably held by the resin 55 disposed not only in the insertion hole H40 but also in the through-hole H50.

Note that the resin 55 may be injected after the optical fiber 30 is inserted into the insertion hole H40. The resin 55 may be injected into the through-hole H50.

When the end portion of the wire 49 of the ferrule 40 is solder-bonded to the second bonding electrode 69 of the wiring board 60, a force for separating a side surface rear part of the ferrule 40 from the front surface 60SA of the wiring board 60 is generated by surface tension or the like of melted solder. However, the ferrule 40 and the guide member 50 are produced by cutting a bonded wafer 45. Further, a side surface of the ferrule 40 and a side surface of the guide member 50 are the same cut surface and are in a state in which the side surfaces are located on the same plane. Both the side surfaces are bonded to the wiring board 60. Accordingly, the ferrule 40 is not detached from the wiring board 60 by the surface tension of the melted solder. Therefore, reliability of the image pickup apparatus 1 is high. Further, the side surface of the ferrule 40 and the side surface of the guide member 50 are the same cut surface and located on the same plane. Both the side surfaces are bonded to the wiring board 60. Accordingly, reliability of electric connection of the end portion of the wire 49 and the second bonding electrode 69 is high.

### MODIFICATIONS

Image pickup apparatuses for endoscope 1A to 1E in modifications are similar to the image pickup apparatus 1 and have the same effects as the effects of the image pickup apparatus 1. Therefore, components having the same functions are denoted by the same reference numerals and signs and explanation about the components is omitted.

### Modification 1

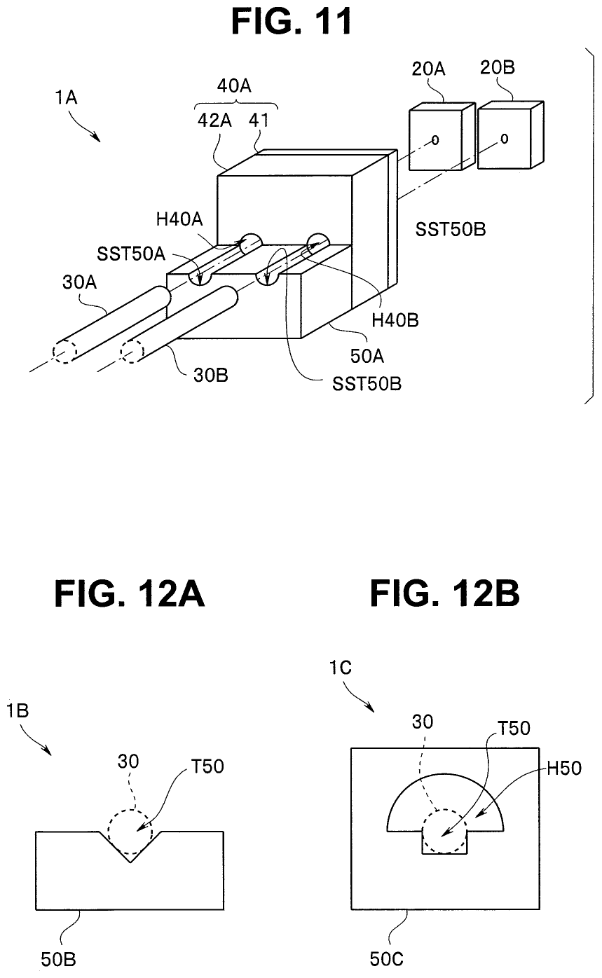

As shown in FIG. 11, the image pickup apparatus for endoscope 1A in a modification 1 includes a plurality of optical fibers 30A and 30B and a plurality of optical elements 20A and 20B. A plurality of insertion holes H40A and H40B are formed in a ferrule 40A. A guide member 50A includes a plurality of guide surfaces SST50A and SST50B.

The guide member 50A of the image pickup apparatus 1A includes the guide surfaces SST50A and SST50B not on wall surfaces but on side surfaces of through-holes. Like the image pickup apparatus 1 shown in FIG. 10, the image pickup apparatus 1A is produced by cutting the bonded wafer including the through-hole H50. However, the bonded wafer including a frame portion is cut by a cutting line extending across an opening of the through-hole H50. Accordingly, a frame portion having the same external dimension as the external dimension of the ferrule 40A is absent in the cut guide member 50A.

### Modifications 2 and 3

As shown in FIG. 12A, the guide surface SST50 of the image pickup apparatus for endoscope 1B in a modification 2 is a wall surface of a V groove. As shown in FIG. 12B, the guide surface SST50 of the image pickup apparatus for endoscope 1C in a modification 3 is a wall surface and a bottom surface of a rectangular groove.

In other words, a sectional shape of the guide surface SST50 may be a circular shape, a V shape, or a rectangular shape if the guide surface SST50 functions as a guide when the optical fiber 30 is inserted into the insertion hole H40. Further, a guide member may not include a frame portion configuring a through-hole. Note that the through-hole H50 of the image pickup apparatus 1C is substantially semicircular.

### Modifications 4 and 5

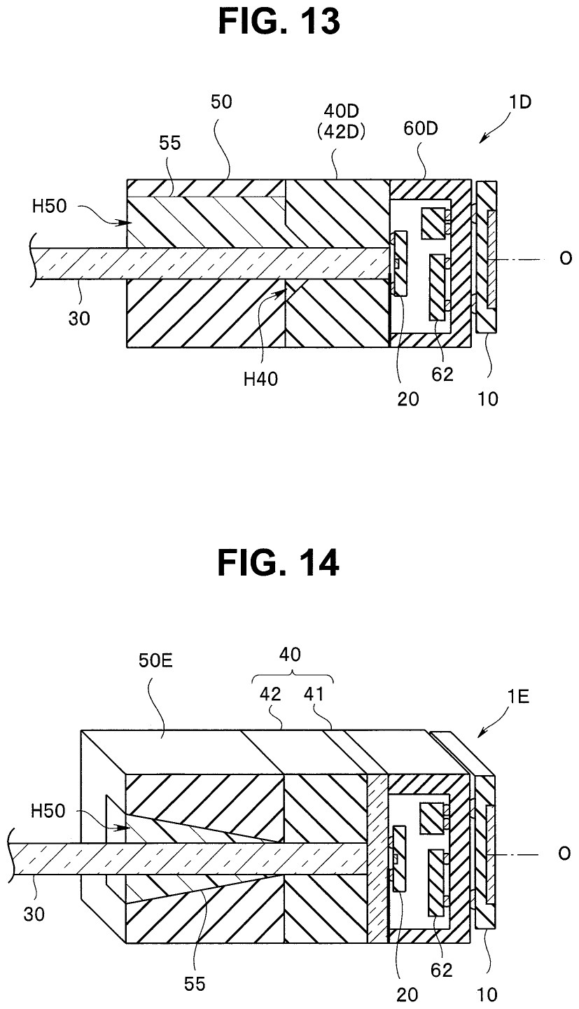

As shown in FIG. 13, the image pickup apparatus for endoscope 1D in a modification 4 is a so-called vertical type in which a light receiving surface of the image pickup device 10 is orthogonal to an optical axis. A wiring board 60D is a three-dimensional wiring board.

A ferrule 40D does not include a glass plate. In other words, the insertion hole H40 is a through-hole. Further, a taper is formed in the insertion hole H40.

As shown in FIG. 14, in the image pickup apparatus for endoscope 1E in a modification 5, in a guide member 50E, an entire inner surface of the through-hole H50, in which a taper is formed, is a guide surface. Since an opening portion of the through-hole H50 is large, it is easy to insert the optical fiber 30 into the image pickup apparatus 1E.

Note that various forms can be applied to the guide surface of the guide member. For example, when an insertion direction of the optical fiber is inclined with respect to the optical axis, a center line of the groove may be inclined in the same manner as the insertion direction.

The ferrule to which the guide member that supports the insertion of the optical fiber is bonded is applied to image pickup apparatuses having various structures. It goes without saying that an O/E type image pickup apparatus in which an optical element is a light receiving element including a light receiving unit such as a photodiode has the same effects as the effects of the image pickup apparatus 1. In other words, the optical element only has to emit or receive an optical signal. The image pickup apparatus in the embodiment may include a light emitting element and a light receiving element.

The image pickup apparatus of the present invention may include pluralities of the components included in each of the image pickup apparatuses 1A to 1E. It goes without saying that endoscopes 9A to 9E including the image pickup apparatuses 1A to 1E have the effects of the endoscope 9 and further have the effects of each of the image pickup apparatuses 1A to 1E.

The present invention is not limited to the embodiment, the modifications, and the like explained above. Various changes, combinations, and applications are possible within a range not departing from the gist of the invention.

What is claimed is:

1. A manufacturing method for an image pickup apparatus for endoscope, the manufacturing method comprising:
forming, in a first wafer, a plurality of insertion holes into which a plurality of optical fibers are individually inserted and forming, in a second wafer, a plurality of through-holes each including, on a wall surface, a guide surface functioning as a guide in inserting the plurality of optical fibers individually into respective holes of the plurality of insertion holes;
bonding the first wafer and the second wafer to produce a bonded wafer;
cutting the bonded wafer to thereby produce ferrules to which guide members each including the guide surface are bonded; and
mounting an optical element on each of the ferrules and fixing the optical fibers with resin in a state in which the optical fibers are inserted into the insertion holes by passing through the respective guide members.

2. The manufacturing method for the image pickup apparatus for endoscope according to claim 1, wherein
a glass wafer including a first principal surface and a first silicon wafer including a second principal surface are bonded to produce the first wafer, and the insertion holes of the first wafer include the glass wafer as bottom surfaces, and
the second wafer is a second silicon wafer.

3. The manufacturing method for the image pickup apparatus for endoscope according to claim 1, wherein the resin is injected into the through-holes when the optical fibers are fixed by the resin.

4. The manufacturing method for the image pickup apparatus for endoscope according to claim 1, wherein, when the bonded wafer is cut, the bonded wafer is cut along a cutting line extending across openings of the through-holes.

5. An image pickup apparatus for endoscope comprising:
an image pickup device configured to output an image pickup signal;
an optical element configured to convert the image pickup signal into an optical signal;
an optical fiber configured to transmit the optical signal;
a ferrule including a first principal surface and a second principal surface on an opposite side of the first principal surface, the optical element being mounted on the first principal surface, the optical fiber being inserted into an insertion hole with an opening on the second principal surface; and
a guide member including a third principal surface and a fourth principal surface on an opposite side of the third principal surface, the third principal surface being bonded to the second principal surface, the guide member including a guide surface extending from an inner surface of the insertion hole.

6. The image pickup apparatus for endoscope according to claim 5, wherein the guide surface is a wall surface of a through-hole formed in the guide member.

7. The image pickup apparatus for endoscope according to claim 6, wherein an opening of the through-hole is substantially rectangular or substantially semicircular.

8. The image pickup apparatus for endoscope according to claim 5, further comprising a wiring board including a front surface to which a side surface of the ferrule and a side surface of the guide member are bonded, wherein
the ferrule includes, on the first principal surface, a first bonding electrode on which the optical element is mounted and a wire extending from the first bonding electrode, and
an end portion of the wire and a second bonding electrode of the wiring board are bonded by solder.

9. The image pickup apparatus for endoscope according to claim 5, wherein the ferrule and the guide member are made of silicon.

10. The image pickup apparatus for endoscope according to claim 5, wherein the image pickup apparatus includes:
a plurality of optical fibers;
a plurality of optical elements;
the ferrule in which a plurality of insertion holes are formed; and
the guide member including a plurality of guide surfaces.

11. An endoscope comprising:
an image pickup device configured to output an image pickup signal,
an optical element configured to convert the image pickup signal into an optical signal;
an optical fiber configured to transmit the optical signal;
a ferrule including a first principal surface and a second principal surface on an opposite side of the first principal surface, the optical element being mounted on the first principal surface, the optical fiber being inserted into an insertion hole with an opening on the second principal surface; and
a guide member including a third principal surface and a fourth principal surface on an opposite side of the third principal surface, the third principal surface being bonded to the second principal surface, the guide member including a guide surface extending from an inner surface of the insertion hole.
